(19) United States
(12) Patent Application Publication
(10) Pub. No.: US 2023/0385678 A1
(43) Pub. Date: Nov. 30, 2023

(54) METHOD FOR FULL QUANTUM MECHANICAL SIMULATION FOR REAL SYSTEMS

(71) Applicant: SOPHYICS TECHNOLOGY, LLC, Mclean, VA (US)

(72) Inventors: Xindong WANG, Dayton, MD (US); Qiang FU, Mclean, VA (US)

(73) Assignees: SOPHYICS TECHNOLOGY, LLC, Mclean, VA (US); KINGSTONE INVESTMENT, LLC, Mclean, VA (US)

(21) Appl. No.: 18/249,567

(22) PCT Filed: Oct. 21, 2021
(86) PCT No.: PCT/US2021/056099

§ 371 Date: Apr. 19, 2023

Related U.S. Application Data
(60) Provisional application No. 63/104,122, filed on Oct. 22, 2020.

(51) Int. Cl. G06N 10/60; G06N 10/20

(52) U.S. Cl. CPC G06N 10/60; G06N 10/20

(57) ABSTRACT

A computer-implemented method for solving low energy excitation spectrum and their corresponding eigenstates is provided. The eigenstates solved include ground state and single-fermion excited states. The method includes: calculating ground state and single-fermion excited states of a system and/or a sub-system of particles in isolation; calculating a coupling between fermions using a quantum mechanical hopping matrix elements between hybridized fermions and long range Coulomb and exchange interactions for a given charge and spin density; calculating a system free energy as a function of structural properties of molecules based on an energy spectrum that depends on positions of the particles and orientations of the particles, the positions being the center of charge for each of the particles; and simulating the systems of particles by integrating a time evolution of the structural properties using full quantum mechanical time evolution of a quantum state given the initial state.

### CROSS-REFERENCE TO RELATED APPLICATIONS

This application claims the benefit of priority to U.S. Provisional Application No. 63/104,122, filed on Oct. 22, 2020, the entire contents of which are incorporated herein by reference.

### INTRODUCTION

The present disclosure is applicable in the field of first principles quantum mechanical computation of material properties. The present disclosure provides computing methods for predicting the structure and function of, and interaction among, molecules, atoms, nuclei and other particles, including biomolecules in an environment, by means of applying a novel method to solve the exact quantum mechanical eigenstates and eigenvalues of many-body systems with computation time that scales in polynomial power with the number of particles under study.

In all existing methods, the nuclei in the system are treated classically, using the Born-Oppenheimer approximation and only electrons are treated quantum mechanically. So in a sense, they are all semi-quantum mechanical. The present disclosure provides a novel method that treats all nuclei and the electrons as quantum mechanical particles, previously considered totally infeasible. The practical use of the method is mainly due to its polynomial time scaling for the accurate solution of the low energy eigenstates of a whole system.

### SUMMARY

According to an embodiment of the present disclosure, a computer-implemented method for solving low energy excitation spectrum is provided. The low energy excitation spectrum includes a ground state energy and corresponding eigenstates of physical properties of systems of particles, including ground state |Vac_{γ} [symbol] and single-fermion excited states \hat{γ}_{μ}^{†}|Vac [symbol] . The method comprises: calculating ground state and single-fermion excited states of a system and/or a subsystem of particles in isolation; calculating a coupling between fermions using a quantum mechanical hopping matrix elements between hybridized fermions and long range Coulomb and exchange interactions for a given charge and spin density; calculating a system free energy as a function of structural properties of molecules based on the solved energy spectrum of the system, given positions of the particles and orientations of the particles, the positions being the center of charge for each of the particles; and simulating the systems of particles by integrating a time evolution of the structural properties using the time evolution of the quantum state given its initial state.

According to an embodiment of the present disclosure, a computer implemented method for simulating interactions of particles is provided. The method includes receiving initial conditions of particles under simulation; calculating an initial isolated state for each of the particles based on the corresponding initial conditions; calculating an initial system state, the initial system state being a tensor product of the initial isolated state of each particle at an initial position of each of the corresponding particle; calculating a projection coefficient based on the initial system state and eigenstate of the total Hamiltonian of the particles; simulating an evolution of a time dependent full quantum state of the particles based on the projection coefficent; and obtaining expectation values of the particles based on the time dependent full quantum state of the particles. A computation time of simulating the evolution of time dependent full quantum state of the particles scales in polynomial power with the number of particles.

### BRIEF DESCRIPTION OF THE DRAWINGS

FIG. 1 depicts a flow chart of a computational algorithm, consistent with the disclosed embodiments.

FIG. 2 depicts a flow chart of an exemplary process of full quantum mechanical simulation of real systems by a computerized system, consistent with the disclosed embodiments.

### DESCRIPTION

### TECHNICAL FIELD

The present disclosure is applicable in the field of first principles quantum mechanical computation of material properties. The method of the disclosure is implemented in a computer or any suitable programmable electronic equipment, and provides the effect or advantage of reducing molecular dynamics simulation time by a novel algorithm that treats all nuclei and electrons in the system as quantum mechanical particles of different type and all dynamical responses of the system at elevated temperatures are modeled as full quantum mechanical responses.

### BACKGROUND

Simulating a system of interacting atoms, such as large biomolecules in a solvent, is critical for designing drugs, designing new materials, among others. Direct molecular dynamics (MD) simulation with ab initio quantum mechanical and molecular mechanical (QM/MM) methods is very powerful for studying the mechanism of chemical reactions in a complex environment. However, current state-of-the-art computer implemented method which is capable of exact diagonalization of the many body Hamiltonian scales exponentially in computational time with the number of particles under study, and thus are very time consuming and impractical for large systems. The computational cost of QM/MM calculations during MD simulations can be reduced significantly using semi-empirical QM/MM methods, but the accuracy of these semi-empirical methods is lower, and more fundamentally they are not capable of capturing the effect of the off-diagonal quantum entanglement effects of the fermions in the system, most notably, the entanglement of protons, that are crucial in understanding bio-molecular systems at room temperature. It has been a holy grail for scientists to have a first principles full quantum mechanical description of any system under study since the discovery of quantum mechanics almost one century ago. For a real system, as long as we have a Hamiltonian that properly captures all the important interactions between quantum particles (fermions in particular), then the ultimate task at hand is to solve for the eigenstates and eigenvalues of the Hamiltonian, especially for the states close to the ground state, which is defined as the lowest energy state of the Hamiltonian. Once these eigenstates and eigenvalues are known for the system, the physical properties of the system, including its dynamic evolutions, are known, given its initial condition, because of Schrodinger equation:

$$i ℏ \frac{\partial }{\partial t} \vert Ψ ( t ) 〉 = \hat{H} \vert Ψ ( t ) 〉 \qquad ( 1 )$$

Once the wavefunction is known, per quantum mechanics prescription, any physical observable, when measured in the state of |Ψ(t) [symbol] will be the expectation of the operator corresponding to that observable, i.e. [symbol] Ψ(t)|Ô|Ψ(t) [symbol] . The Schrodinger equation can be integrated given the initial condition, expanded as a linear combination of the eigenstates |ψᵢ [symbol] of the system

$$\vert Ψ ( 0 ) 〉 = {\sum }_{i} {c}_{i} \vert {ψ}_{i} 〉 \qquad ( 2 )$$

such that

$$\vert Ψ ( t ) 〉 = {\sum }_{i} {c}_{i} {e}^{- i {E}_{i} t} \vert {ψ}_{i} 〉 \qquad ( 3 )$$

Thus the evolution of the system is known if the eigenstate |ψᵢ [symbol] are solved by the following eigenvalue problem

Ĥ|ψᵢ [symbol] =Eᵢ|ψᵢ [symbol] (4)

And the physical observables can be predicted/calculated from first principles. The above can easily be generalized to an ensemble of initial states where the initial condition is specified by the density matrix. A general many-body fermionic Hamiltonian is of the following form

$$\hat{H} = {\sum }_{i , j} {h}_{ij} {\hat{c}}_{i}^{†} {\hat{c}}_{j} + {\hat{H}}_{2} \qquad ( 5 )$$

where

$${\hat{H}}_{2} = {\sum }_{p q r s} {𝒥}_{p q r s} {\hat{c}}_{p}^{†} {\hat{c}}_{q}^{†} {\hat{c}}_{r} {\hat{c}}_{s} , \qquad ( 6 )$$

One of the main hurdles in achieving this ultimate feast of applying the theory to all matters of human concern is that in quantum mechanics, the degree of freedom in the states scales exponentially with the size of the system and the size of the matrix for the Hamiltonian is thus believed to grow exponentially as well, due to the non-quadratic terms embodied by the 2-body interaction term Ĥ₂. Thus, the consensus is to avoid this exponential catastrophe by making various approximation with varying degree of successes, depending on which area one is interested in.

### DETAILED DESCRIPTION

The present disclosure provides a solution to the aforementioned problem by means of applying a novel method in the simulation to reach the exact quantum mechanical solution of many-body systems with computation time that scales in polynomial power with the number of particles under study. In some embodiments of present disclosure, there is provided a realization of a local “vacuum” state at each local site, and observables that can be measured are due to the inter-site coupling of the particle excitations from these local vacuum state. We assume all the fundamental quantum particles are fermions (bosonic nuclei are and can be modeled as composite fermions). And once the low energy spectrum and the corresponding eigenstates are known for a system, physical properties of the system can thus be derived from first principles. For any fermionic system, we can decompose the full Hilbert space into the tensor product of the two sub-Hilbert space, S⊗E, where S is a single band fermionic system at spatial location ϰ, and E is the rest of the system. For each ϰ, we have the following complete set of orthonormal local states at ϰ, defined by the fermion creation operators

|0 [symbol] , ψ_{↑}^{†}(ϰ)|0 [symbol] , ψ_{↓}^{†}(ϰ)|0 [symbol] , ψ_{↑}^{†}(ϰ)ψ_{↓}^{†}(ϰ)|0 [symbol] (7)
We define the following Bogolyubov vacuum at ϰ as

|Vac(ϰ))=α(ϰ)|0 [symbol] +β(ϰ)\hat{ψ}_{↑}^{†}(ϰ)\hat{ψ}_{↓}^{†}(ϰ)|0 [symbol] (8)

where α(ϰ) and β(ϰ) are c-numbers and

|α(ϰ)|²+|β(ϰ)|²=1, 2|β(ϰ)|²=n_{Vac}(ϰ), (9)

where n_{Vac}(ϰ) is the density of particles at ϰ of the non-empty Bogolyubov vacuum. We further define the following Bogolyubov transformed operators

\hat{ξ}_{↓}(ϰ)=β(ϰ)\hat{ψ}_{↑}^{†}(ϰ)+α(ϰ)\hat{ψ}_{↓}(ϰ),

\hat{ξ}_{↓}⁵⁵⁴ (ϰ)=β*(ϰ)\hat{ψ}^{↑}(ϰ)+α*(ϰ)\hat{ψ}_{↓}^{†}(ϰ),

\hat{ξ}_{↑}(ϰ)=β(ϰ)\hat{ψ}_{↓}^{†}(ϰ)−α(ϰ)\hat{ψ}^{↑}(ϰ),

\hat{ξ}_{↑}⁵⁵⁴ (ϰ)=β*(ϰ)\hat{ψ}_{↓}(ϰ)−α*(ϰ)\hat{ψ}_{↑}^{†}(ϰ). (10)

We further verify the following:

\hat{ξ}_{σ}(ϰ)|Vac(ϰ) [symbol] =0,

\hat{ξ}_{σ}^{†}(ϰ)|Vac(ϰ) [symbol] =−σ\hat{ψ}_{σ}^{†}(ϰ)|0 [symbol] =−σ|ϰσ [symbol] (11)

and the following anti-commutation relations

{\hat{ξ}_{σ}^{†}(ϰ),\hat{ξ}_{σ′}(ϰ′)}=δ_{ϰ,ϰ′}δ_{94 ,σ′} (12)

{\hat{ξ}_{σ}(ϰ),\hat{ξ}_{σ′}(ϰ′)}={\hat{ξ}_{σ}^{†}(ϰ),\hat{ξ}_{σ′}^{†}(ϰ′)}=0. (13)

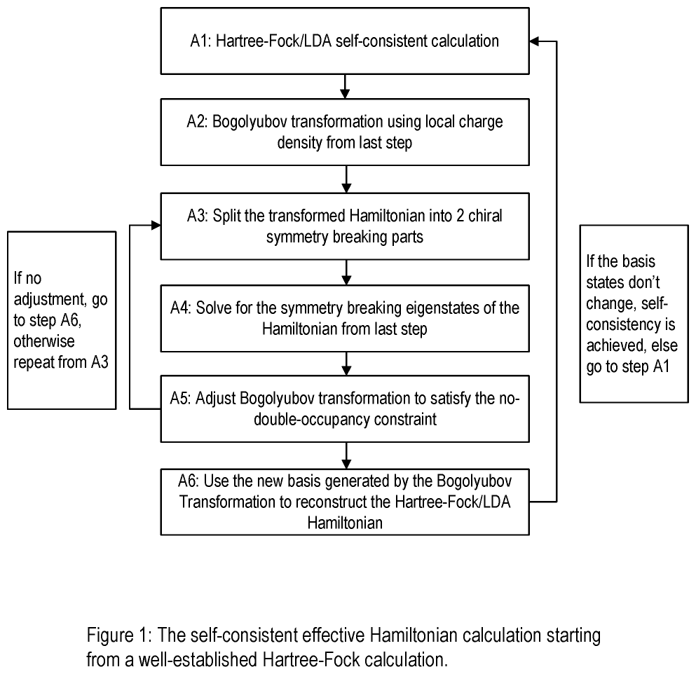

We note that ξ_{σ}^{†}(ϰ) creates a fermion from |Vac(ϰ) [symbol] with a fractional charge of |e|(|α|²−|β|²)=|e|(1−2|β|²) and spin σ. The transformation effectively defines a general class of fermions, b-fermions, that become electrons when β=0, positrons when α=0, and Majorana fermions when |β|²=½.
The self-consistent effective Hamiltonian theory asserts that for any interacting many-body fermionic system, there exists two chiral symmetry broken effective Hamiltonian Ĥ_{eff}⁽⁺⁾, and Ĥ_{eff}⁽⁻⁾ that are quadratic and each provides a set of eigenstate as in Eq. (4), and furthermore these eigenstate gives the exact ground state and single-fermion excitation states of the full Hamiltonian (5). Since it is known that a quadratic Hamiltonian corresponds to a polynomial scaling in terms of degree of freedoms which is proportional to the number of sites ϰ, as opposed to the exponential scaling, the current disclosure provides a technological break-through in the computational algorithm for exact quantum mechanical many-body solutions that avoids the errors made by the various approximations that neglected off-diagonal long-range order due to the neglect of off-diagonal paring terms that the continuous Bogolyubov transformation enables mathematically. This technological breakthrough also enables computer simulations of dynamical processes of large systems instead of performing resource consuming, high risk trial by error experiments in lab.
The following gives a detailed description of the self-consistent loop in our algorithm. A flow chart of the algorithm is illustrated in FIG. 1.

### 1. A1: Set Up Hartree-Fock Mean Field Hamiltonian Parameters

For self-consistent Hartree-Fock calculation, the following quadratic Hamiltonian is established and solved

$${\hat{H}}_{HF} = {\sum }_{μ} {E}_{μ} {\sum }_{σ} {\hat{ψ}}_{μ σ}^{†} {\hat{ψ}}_{μ σ} \qquad ( 14 )$$

$${\hat{ψ}}_{μ σ} = {\sum }_{i} {v}_{μσ , i} {\hat{c}}_{i}$$

$$\vert {Vac}_{HF} 〉 = {\prod }_{{E}_{μ} < 0 , σ} {\hat{ψ}}_{μ σ}^{†} \vert 0 〉$$

i.e., all E_{μ} and υ_{μσ,i} have been found at the conclusion of Hartree-Fock.
   2. A2: Bogolyubov transformation
The quadratic Hartree-Fock Hamiltonian will double in matrix size whenever the Bogolyubov transformation is introduced

$${\hat{H}}_{HF} = {( \begin{matrix}{\hat{ξ}}_{↑}^{†} \\ {\hat{ξ}}_{↓}^{†} \\ {\hat{ξ}}_{↑} \\ {\hat{ξ}}_{↓}\end{matrix} )}^{T} ( \begin{matrix}{\hat{h}}_{↑ ↑} & 0 & 0 & {Δ}_{↑ ↓} \\ 0 & {\hat{h}}_{↓ ↓} & {Δ}_{↓ ↑} & 0 \\ 0 & {Δ}_{↓ ↑}^{†} & 0 & 0 \\ {Δ}_{↑ ↓}^{†} & 0 & 0 & 0\end{matrix} ) ( \begin{matrix}{\hat{ξ}}_{↑} \\ {\hat{ξ}}_{↓} \\ {\hat{ξ}}_{↑}^{†} \\ {\hat{ξ}}_{↓}^{†}\end{matrix} ) \qquad ( 15 )$$

where \hat{ξ}_{σ} is understood to be a vector of \hat{ξ}_{iσ}.
   3. A3: Splitting of the Hamiltonian into Chiral Symmetry Breaking Parts
We break the above qudratic Hamiltonian into two parts.

$${\hat{H}}_{HF} = {\hat{H}}^{( + )} + {\hat{H}}^{( - )} \qquad ( 16 )$$

$$where$$

$${\hat{H}}^{( + )} = {( \begin{matrix}{\hat{ξ}}_{↑}^{†} \\ {\hat{ξ}}_{↓}^{†} \\ {\hat{ξ}}_{↑} \\ {\hat{ξ}}_{↓}\end{matrix} )}^{T} ( \begin{matrix}{\hat{h}}_{↑ ↑} & 0 & 0 & {Δ}_{↑ ↓} \\ 0 & 0 & 0 & 0 \\ 0 & 0 & 0 & 0 \\ {Δ}_{↑ ↓}^{†} & 0 & 0 & 0\end{matrix} ) ( \begin{matrix}{\hat{ξ}}_{↑} \\ {\hat{ξ}}_{↓} \\ {ξ}_{↑}^{†} \\ {\hat{ξ}}_{↓}^{†}\end{matrix} ) \qquad ( 17 )$$

$$and$$

$${\hat{H}}^{( - )} = {( \begin{matrix}{\hat{ξ}}_{↑}^{†} \\ {\hat{ξ}}_{↓}^{†} \\ {\hat{ξ}}_{↑} \\ {\hat{ξ}}_{↓}\end{matrix} )}^{T} ( \begin{matrix}0 & 0 & 0 & 0 \\ 0 & {\hat{h}}_{↓ ↓} & {Δ}_{↓ ↑} & 0 \\ 0 & {Δ}_{↓ ↑}^{†} & 0 & 0 \\ 0 & 0 & 0 & 0\end{matrix} ) ( \begin{matrix}{\hat{ξ}}_{↑} \\ {\hat{ξ}}_{↓} \\ {ξ}_{↑}^{†} \\ {ξ}_{↓}^{†}\end{matrix} ) \qquad ( 18 )$$

### 4. A4: Solve for the Eigenstates of the CHIRAL SYMMETRY BREAKING HAMILTONIAN

We solve for the ground state of the chiral symmetry breaking Hamiltonian with the charge conservation constraint. (Note that in the original Hartree-Fock calculation, chemical potential does not need to be introduced as \hat{N} is a good quantum number.

$$\hat{ℋ} = {\hat{H}}^{( + )} - δ {\sum }_{i} {\hat{c}}_{i}^{†} {\hat{c}}_{i} \qquad ( 19 )$$

Note that the above self-consistent calculation seemingly take the complete spin-polarized starting Hartree-Fock as one of the solutions, except for the fact the total charge does not satisfy the charge conservation condition. The chemical potential δ is introduced as a Lagrangian multiplier due to the constraint.
   A5: Imposing no-double-occupancy constraint
The ground state of a quadratic Hamiltonian is known to be of the form

$$\begin{matrix}\vert {0}_{γ} 〉 = {N}_{γ} exp \{ {\sum }_{ϰ σ , {ϰ}^{′} {σ}^{′}} \frac{1}{2} ( {λ}_{ϰ σ , {ϰ}^{′} {σ}^{′}} {\hat{ξ}}_{ϰ σ}^{†} {\hat{ξ}}_{{ϰ}^{′} {σ}^{′}}^{†} ) \} \vert {0}_{ξ} 〉 \\ = {N}_{γ} {\prod }_{〈 ϰ σ , {ϰ}^{′} {σ}^{′} 〉} \{ 1 + {λ}_{ϰσ , {ϰ}^{′} {σ}^{′}} {\hat{ξ}}_{ϰσ}^{†} {\hat{ξ}}_{{ϰ}^{′} {σ}^{′}}^{†} \} \vert {0}_{ξ} 〉\end{matrix} \qquad ( 20 )$$

$$where$$

$${λ}_{ϰσ , {ϰ}^{′} {σ}^{′}} = - {λ}_{{ϰ}^{′} {σ}^{′} , ϰσ}$$

is anti-symmetric and [symbol] _{γ} is a normalization constant such that

[symbol]

0_{γ}|0_{γ} [symbol] =1
And the single particle excited states are given by the diagonalization of the quadratic effective Hamiltonian

$${\hat{H}}_{eff} = {E}_{V a c} + {\sum }_{i} {E}_{i} {\hat{γ}}_{i}^{†} {\hat{γ}}_{i} , , {E}_{i} > 0 , ∀ i \qquad ( 21 )$$

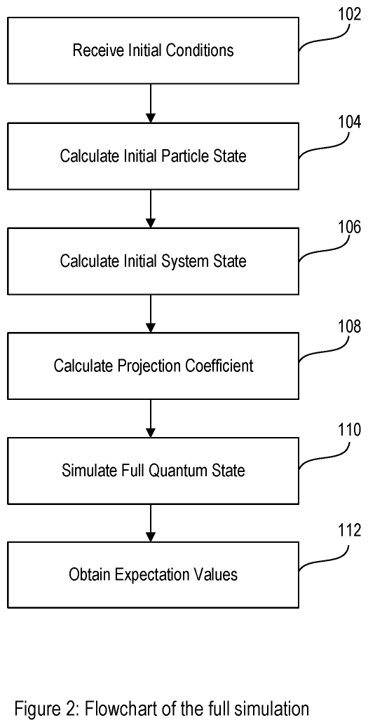

For each of the onsite pairing term in (1+λᵢ\hat{ξ}_{i↑}^{†}\hat{ξ}_{i↓}^{†}), we can further perform additional Bogolyubov transformation. Since additional Bogolyubov transformation will change Ĥ⁽⁺⁾ in Eq. (19), an additional loop is needed.
   6. A6: Construct new Hartree-Fock Hamiltonian from full many-body Hamiltonian in the new chiral symmetry breaking basis
Under the new fermion operators defined with the new chiral symmetry broken vacuum, one can reconstruct the full many-body Hamiltonian and drop those terms that have more annihilation operators than the creation operators. This can be done by direct substitution of the ĉ operators by the inverse Bogolyubov transformation. And because of the no-double-occupancy constraint, the onsite Hubbard U will not be present, i.e., it is renormalized away. If the new basis converges, then the self-consistent calculation is done, otherwise, go to step A1.
According to an embodiment of the present disclosure, a computer-implemented method for solving low energy excitation spectrum is provided. The low energy excitation spectrum includes a ground state energy and corresponding eigenstates of physical properties of systems of particles, including ground state |Vac₆₅ [symbol] and a single-fermion excited states \hat{γ}_{μ}^{†}|Vac [symbol] .
The method includes calculating ground state and single-fermion excited states of a system and/or a subsystem of particles in isolation. The calculation steps are described in the algorithm detailed in A1 to A6 and depicted in FIG. 1.
The method also includes calculating a coupling between fermions using a quantum mechanical hopping matrix elements between hybridized fermions and long range Coulomb and exchange interactions for a given charge and spin density. Coupling matrix elements may be updated using Hartree-Fock calculations whenever single-particle basis states are updated due to Bogolyubov transformation. Here the hybridized fermions also referred to the mixing of particle and anti-particle/hole states.
The method further includes calculating a system free energy as a function of structural properties of molecules based on the solved energy spectrum of the system, given positions of the particles and orientations of the particles, the positions being the center of charge for each of the particles.
The method further includes simulating the systems of particles by integrating a time evolution of the structural properties using time evolution of the quantum state given its initial state.
Here, the simulated systems of particles include at least one of a gas, a liquid, a nano-device, biomolecules such as proteins, RNA, and/or DNA, as well as polymers and small molecules.
In some embodiments, the method for solving low energy excitation spectrum may further include identifying a number of coherent of-diagonal long-range ordered quantum states at room temperature. The coherent quantum states are building blocks of qubits for quantum computer and quantum memory storage. Here the coherent quantum states are identified by the singular values of the anti-symmetric off-diagonal matrix λ_{ϰσ,ϰ′σ′} in Eq. (20).
In some embodiments, the simulated systems of particles may include a molecule or molecules for designing a new material. The method may further include entering data corresponding to a designed material; upon completion of the simulation, generating data relating to positions, velocities, energies of the molecule or molecules; and estimating macroscopic properties of the molecule or molecules and validity of the designed material.
In some embodiments, the method for solving low energy excitation spectrum may further include identifying energy transfer channels and/or frequencies during bond formation and/or breaking between particles. Here the energy transfer channels are identified by the off-diagonal long range paring terms λ_{ϰσ,ϰ′σ′} in Eq. (20).
In some embodiments, the identifying energy transfer channels and/or frequencies are used for designing low intrusion method treatments. The method may further include targeting electrical signals in the identified channel and/or frequency to enhance and/or impede the bond formation and/or breaking. Here the energy transfer channels are identified by the off-diagonal long range paring terms λ_{ϰσ,ϰ′σ′} in Eq. (20). In our design, whenever the energy transfer is interfered by the targeting electrical signals, the corresponding off-diagonal long-range ordering will be impacted.
In some embodiments, the simulated systems of particles may include a molecule or molecules for designing a new drug. The method may further include designing drugs against a drug target; selecting a new drug; entering data corresponding to the selected new drug; upon completion of the simulation, generating data relating to positions, velocities, energies of the molecules; estimating macroscopic properties of the molecules and validity of the selected drug; and estimating a potency of the selected drug in enhancement or impediment of bond formation between bio-molecules such as protein molecules.
FIG. 2 depicts a flow chart of an exemplary process of full quantum mechanical simulation of real systems by a computerized system, consistent with the disclosed embodiments. In these steps, whenever the calculation of states are mentioned, they are referring to the computational process embodied by the algorithm as described above and depicted in FIG. 1. Also the singular values of the anti-symmetric off-diagonal pairing matrix λ_{ϰσ,ϰ′σ′} in the ground state (20) are to be identified with the energy transfer channels for the corresponding off-diagonal long-range ordered quantum state.
In step 102, the computerized system receives initial conditions of particles to be simulated. Particles may refer to mathematical representation of micro physical objects, such as atoms, nuclei, molecules, electrons, and/or other similar molecular, atomic, or sub-atomic objects. Initial conditions of a particle may refer to parameters of a particle at an initial time. For example, an initial time may be a time at beginning of simulation. Examples of initial conditions may include an initial mass, velocity, orientation, location, energy level, quantum state, polarity, parity, and/or other physical properties of a particle.
In step 104, the computerized system calculates an initial particle state. In some embodiments, the computerized system may calculate an initial particle state for each of the particles included in the simulation. The initial particle state may represent a physical state of a particle in isolation of (not considering) other particles in the simulation. The computerized system may calculate the initial particle state based on the initial conditions of the particle. For example, the initial state for each particle in isolation may be calculated by obtaining an equilibrium state (or ground state) where a first excited state has a large gap. In another example, the initial particle state may be calculated as a thermo-distribution per Boltzmann distribution.
In step 106, the computerized system calculates an initial system state. In some embodiments, the computerized system may calculate the initial system state by calculating a tensor product of the initial particle state of each particle at an initial position of each of the corresponding particle. The initial position of a particle may be a position of the center of mass of the corresponding the particle, at the beginning of the simulation. In some embodiments, the initial position may be included as part of the initial conditions.
In step 108, the computerized system calculates projection coefficients. Since the initial system state calculated in step 106 is not an eigenstate of the total Hamiltonian, the eigenstates of the total Hamiltonian needs to be obtained. In some embodiments, the computerized system may project the initial system state obtained in step 106 into the eigenstates of the Hamiltonian to get the projection coefficients. For example, the computerized system may perform the projection by calculating an inner product of quantum mechanical Hilbert space vectors corresponding to 1) the initial system state and 2) the eigenstates of the full Hamiltonian, the solution of which is at the heart of this disclosure.
In step 110, the computerized system simulates an evolution of a time dependent full quantum state of the particles. Based on the projection coefficient calculated in step 108, the full Hamiltonian of the particles can be obtained. The computerized system may then derive the full quantum state of the particles a given time. In some embodiments, the computerized system may perform simulation of the evolution of the particles over a period of time using the time evolution of the quantum state given its initial state.
In step 112, the computer system obtains expected values of the particles. Once simulated results of the full quantum state of the particles are obtained, the computerized system may thus obtain expectation values of various parameters relevant to the physical state of the particles. Examples of the expectation values may include mass, velocity, orientation, location, energy level, and/or other physical properties of the particles or the system of particles.
It is important to point out that the size of the matrix scales linearly with the number of sites times and the number of bands/types of fermions in the system, i.e., Nₐₜₒₘₛ×N_{orbitals}, not exponential as other full quantum mechanical methods.
The disclosure achieves the technical advantage or effect of performing complex molecular dynamics simulations with great speed, while using computers with a conventional computing capacity.
Another aspect of the disclosure relates to a piece of equipment or a system for simulating systems of atoms. The computer comprises data input means, data output means, a processor and a computer program stored in the computer memory.
The method as disclosed herein applied to the field of quantum computer enables finding the candidate material that exhibits many long quantum coherence times at room temperature.
The method as disclosed herein can be applied to the field of material design: the aforementioned method is applied to solve for the complete eigenstates and eigenvalues of the system under study, the interested properties of the material can be derived, thus the method enables testing a greater amount of material compositions, and therefore increases the possibility of finding useful material with targeted properties.
The method as disclosed herein can be applied to the field of drug design: the aforementioned method is applied to solve for the the complete eigenstates and eigenvalues of the system of molecules under study, the properties of and interactions among the molecules can be derived, thus the method enables testing a greater amount of molecules such as chemical compounds, and therefore increases the possibility of finding useful compounds for treating diseases.
The method as disclosed herein can also be applied to the field of medical procedural design, which enables testing the effect of electromagnetic signal targeting the energy transfer channels in biomolecular bond formation and/or breaking.
Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A computer-implemented method for solving a low energy excitation spectrum, including a ground state energy and the single-fermion excitation energies, and corresponding eigenstates of a system and/or subsystems of particles, including a ground state |Vac_{γ} [symbol] and the single-fermion excited states \hat{γ}_{μ}^{†}|Vac [symbol] , the method comprising:
calculating a ground state and single-fermion excited states of a system and/or a subsystem of particles in isolation;
calculating a coupling between fermions using quantum mechanical hopping matrix elements between hybridized fermions and long range Coulomb and exchange interactions for a given charge and spin density;
calculating a system free energy as a function of structural properties of molecules based on the solved energy spectrum of the system, given positions of the particles and orientations of the particles, the positions being the center of charge for each of the particles; and
simulating the system of particles by integrating a time evolution of the structural properties using the time evolution of the quantum state given its initial state.

2. The computer-implemented method according to claim 1, wherein the simulated systems of particles include at least one of a gas, a liquid, a nano-device, biomolecules such as proteins, RNA, and/or DNA, as well as polymers and small molecules.

3. The computer-implemented method according to claim 1, further comprising:
identifying a number of coherent of-diagonal long-range ordered quantum states at room temperature, wherein the coherent quantum states are building blocks of qubits for quantum computer and quantum memory storage.

4. The computer-implemented method according to claim 1, wherein the simulated systems of particles include a molecule or molecules for designing a new material, and the method further comprising:
entering data corresponding to a designed material;
upon completion of the simulation, generating data relating to positions, velocities, energies of the molecule or molecules; and
estimating macroscopic properties of the molecule or molecules and validity of the designed material.

5. The computer-implemented method according to claim 1, further comprising: identifying energy transfer channels and/or frequencies during bond formation and/or breaking between particles.

6. The computer-implemented method according to claim 5, wherein the identifying the energy transfer channels and/or frequencies are used for designing low intrusion method treatments, the method further comprising:
targeting electrical signals in the identified channel and/or frequency to enhance and/or impede the bond formation and/or breaking.

7. The computer-implemented method according to claim 1, wherein the simulated systems of particles include a molecule or molecules for designing a new drug, the method further comprising:
selecting a new drug;
entering data corresponding to the selected new drug and bio-molecules;
upon completion of the simulation, generating data relating to positions, velocities, energies of the molecules;
estimating macroscopic properties of the molecules of the new drug and validity of the selected drug; and
estimating a potency of the selected drug in enhancement or impediment of bond formation between molecules including bio-molecules such as protein molecules.

8. The computer-implemented method of claim 1, wherein the particles include at least one of atoms, nuclei and molecules.

9. The computer-implemented method of claim 1, wherein the at least one of atoms, nuclei and molecules is treated as quantum mechanical particles.

10. The computer-implemented method of claim 1, wherein the initial isolated state for each particle is an equilibrium state.

11-14. (canceled)

15. The computer-implemented method of claim 1, wherein the calculating the ground state and single-fermion excited states of a system and/or a subsystem of particles in isolation includes:
setting up Hartree-Fock mean field Hamiltonian parameters and solving the Hamiltonian;
performing Bogoliubov transformation on the Hamiltonian;
splitting the Hamiltonian into chiral symmetry breaking parts;
solving the Hamiltonian to obtain eigenstates of the chiral symmetry breaking Hamiltonian;
imposing no-double-occupancy constraint; and
constructing new Hartree-Fock Hamiltonian from full many-body Hamiltonian in the new chiral symmetry breaking basis.
